(19) United States
(12) Patent Application Publication
(10) Pub. No.: US 2016/0143610 A1
(43) Pub. Date: May 26, 2016

(54) MEDICAL IMAGING DEVICE AND METHOD FOR OPERATING A MEDICAL IMAGING DEVICE

(71) Applicants: Thomas Luthardt, Bamberg (DE); Helmut Repp, Erlangen (DE); Robert Sitzmann, Hirschaid (DE)

(72) Inventors: Thomas Luthardt, Bamberg (DE); Helmut Repp, Erlangen (DE); Robert Sitzmann, Hirschaid (DE)

(21) Appl. No.: 14/951,752

(22) Filed: Nov. 25, 2015

(30) Foreign Application Priority Data
Nov. 25, 2014 (DE) 102014224010.7

(51) Int. Cl. A61B 6/00; A61B 5/055; H04L 29/08; A61B 6/03; H04N 1/00

(52) U.S. Cl. CPC A61B 6/56; A61B 6/032; A61B 6/586; H04N 1/00214; H04L 67/12; A61B 5/055

(57) ABSTRACT

A medical imaging device with a contactless data transmission device that registers interference fields acting thereon is provided. The device includes a bit error registration unit of the data transmission apparatus. The bit error registration unit is embodied and programmed to establish the bit error rate of the data transmission apparatus caused by the interference fields, and to output a warning signal when a predeterminable threshold of the bit error rate is exceeded.

This application claims the benefit of DE 10 2014 224 010.7, filed on Nov. 25, 2014, which is hereby incorporated by reference in its entirety.

### FIELD

The present embodiments relate to a medical imaging device with a contactless data transmission device and an associated method for operating a medical imaging device.

### BACKGROUND

One field of application of the present embodiments relates to the data transmission between the rotating part and the stationary part of a computed tomography scanner. During operation of the computed tomography scanner, the data registered by the x-ray detectors is to be transmitted from the rotating part to the stationary part of the computed tomography scanner in order to be processed further there. Relatively large amounts of data are to be transmitted within a short period of time.

Many currently available computed tomography scanners use a contactless “slip ring” device for data transmission, as is known, for example, from U.S. Pat. No. 5,140,696 A. This data transmission device includes a transmission unit at the rotating part and a reception unit at the stationary part. The transmission unit has at least one radiofrequency line connected to the transmitter as a transmission antenna, which is arranged on the circumference of the rotating part of a rotating frame. The reception unit includes a receiver and at least one reception antenna connected to the receiver, which is formed by a short portion of a radiofrequency line. During operation of the computed tomography scanner, the transmission antenna moves past the reception antenna attached to the stationary part with little clearance such that the signals propagating on the transmitting radiofrequency line couple into the reception antenna via the near field.

In the case of contactless data transmission, this is to be designed in a particularly insensitive manner in relation to disturbance radiation (e.g., caused by cellular telephones, Wi-Fi, telemetry, RFID, etc.). In general, this is brought about by complicated shielding measures. The requirements on contactless data transmission are increasing within the scope of redrafting the international standard IEC 60601-1-2 (fourth edition), which, inter alia, considers immunity in relation to interference fields (e.g., electric, magnetic, electromagnetic). However, the standard gives the producers the freedom to define the usage surroundings and the risk evaluation of medical devices themselves.

By way of example, the greatly increased thresholds (e.g., from 3 V/m to 28 V/m for acting electromagnetic fields) may be observed by providing that there is no Wi-Fi or cellular telephone operation in the direct vicinity of the medical device. A note to the user of the medical device is not sufficient here. A measure that provides these surrounding conditions is to be implemented by the producer.

### SUMMARY AND DESCRIPTION

The scope of the present invention is defined solely by the appended claims and is not affected to any degree by the statements within this summary.

The present embodiments may obviate one or more of the drawbacks or limitations in the related art. For example, a medical imaging device and a method for operating a medical imaging device that may identify the influence of acting interference fields are provided.

According to one or more of the present embodiments, a contactless data transmission apparatus is used as a detector of interference fields (e.g., electric, magnetic, electromagnetic). The data transmission is always active, even if only control data or scrambled data packets are transmitted. The bit error rate correlates with the influence of the interference fields (e.g., electric, magnetic, electromagnetic) of the surroundings at the usage location. By monitoring and evaluating the bit error rate, available hardware may be used to design a device that monitors whether there is interference. Therefore, a user may be warned of a risk, and hence, the risk of a malfunction due to interference fields (e.g., electric, magnetic, electromagnetic) may be minimized. Valuable data about the usage surroundings of medical imaging devices are collected.

In one or more of the present embodiments, a medical imaging device includes a contactless data transmission apparatus that registers acting interference fields (e.g., electric, magnetic, electromagnetic). The device includes a bit error registration unit of the data transmission apparatus. The bit error registration unit is embodied and programmed to establish the bit error rate of the data transmission apparatus caused by the interference fields and to output a warning signal when a predeterminable threshold of the bit error rate is exceeded.

As a result of the option of detecting influencing interference fields (e.g., electric, magnetic, electromagnetic) in the surroundings of the medical imaging device, one or more of the present embodiments offer arguments for reducing the test level of 28 V/m to 3 V/m when testing for interference immunity in relation to interference fields of wireless RF communication devices.

Shielding measures may be simplified and/or reduced. The use of determining and evaluating internal error rates for self-testing offers a simple option without additional hardware. The safety during patient operation is increased, just like the reliability during operation. Hardware errors may be distinguished from errors due to influences from the surroundings. Moreover, data from typical usage surroundings of medical imaging devices may be obtained.

In one development, the medical imaging device includes an indication unit that is embodied and programmed to represent the warning signal optically and/or acoustically.

In a further embodiment, the medical imaging device may include a switch-off unit that is embodied and programmed to interrupt the operation of the medical imaging device when the warning signal occurs.

In a further embodiment, the data transmission device may have a stationary and a rotating unit, between which data is transmitted contactlessly.

The medical imaging device may be a computed tomography scanner or a magnetic resonance imaging scanner.

In one or more of the present embodiments, a method for operating a medical imaging device includes a contactless data transmission. The bit errors in the data transmission caused by the influencing interference fields (e.g., electric, magnetic, electromagnetic) are registered, the bit error rate is established, and a warning signal is emitted when a predeterminable threshold of the bit error rate is exceeded.

There may be optical and/or acoustic indication of the warning signal.

In a development of the method, the operation of the medical imaging device may be interrupted when the warning signal occurs.

In a further embodiment, the data transmission may be performed between units that are moved relative to one another.

### BRIEF DESCRIPTION OF THE DRAWINGS

FIG. 1 shows a block diagram of one embodiment of a medical imaging device with a data transmission apparatus; and

FIG. 2 shows a block diagram of one embodiment of the data transmission apparatus.

### DETAILED DESCRIPTION

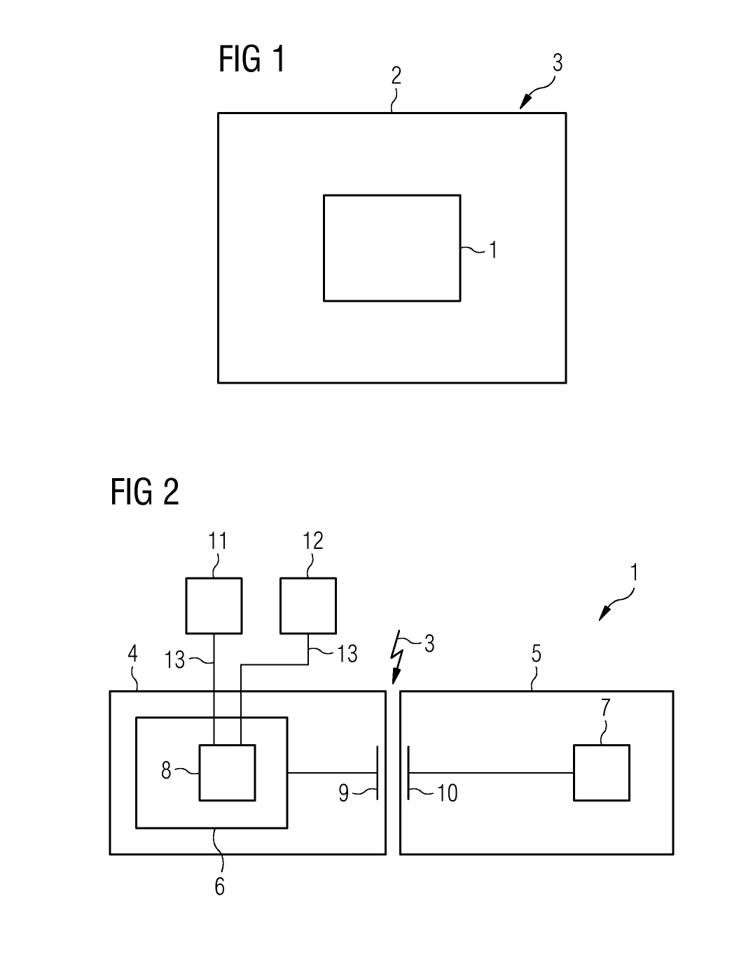

FIG. 1 shows a block diagram of one embodiment of a medical imaging device 2 (e.g., a computed tomography scanner) with a contactless data transmission apparatus 1 for transmitting data (e.g., from an x-ray detector) to an image generation unit not depicted here. The medical imaging device 2 may have a plurality of such contactless data transmission apparatuses. In addition to image data, control data may be transmitted, and data transmission is thus permanently present, even without image recording.

As a result of external interference sources such as, for example, cellular telephones or WLAN routers, an interference field 3 (e.g., electric, magnetic, electromagnetic) may scatter into the contactless data transmission apparatus 1 and thus interfere with the data transmission and hence the operation of the medical imaging device 2.

FIG. 2 shows a block diagram of one embodiment of the data transmission apparatus 1 according to FIG. 1. Useful data and/or control data are transmitted contactlessly between a rotating unit 5 (e.g., a rotating gantry part of a computed tomography scanner with x-ray emitters and x-ray detectors) and a stationary unit 4. The data transmission apparatus 1 includes a reception unit 6 and a transmission unit 7. The transmission unit 7 transmits the data (e.g., image data) to a transmission antenna 10 (e.g., a conductor element with a ring-shaped embodiment). The data transmission apparatus 1 transmits data with a rate of approximately 1 to 5 GBit/s with a CRC and 8B/10B encoding.

The transmission antenna 10 emits electromagnetic radiation, which is received by the reception antenna 9 and forwarded to the reception unit 6. In the contactless transmission path, unwanted interference fields 3 (e.g., electric, magnetic, electromagnetic) may couple into the reception antenna 9 and thus interfere with reception. Therefore, a bit error registration unit 8, which, for example, establishes the bit error rate on the physical layer (e.g., ISO layer model) of the transmission path, is embodied in the reception unit 6. If the bit error rate exceeds a predeterminable threshold, a warning signal 13 is emitted.

The threshold lies at approximately between 10% and 50% of the error threshold at which data transmission is just still possible. The warning signal 13 may be output acoustically or optically on an indication unit 11. With the aid of a switching-off unit 12, the operation of the medical imaging device 2 may be interrupted when the warning signal 13 is present. Alternatively or additionally, the current value of the bit error rate may also be indicated.

The reception antenna 9 therefore also acts as an antenna for interference fields 3 (e.g., electric, magnetic, electromagnetic) that interfere with data transmission (e.g., on the physical layer of the data transmission device 1) and therefore lead to an increased occurrence of bit errors. Instead of increased shielding of the data transmission apparatus 1 so as to make it immune against acting interference fields 3 (e.g., electric, magnetic, electromagnetic), elevated, inadmissible interference fields 3 (e.g., electric, magnetic, electromagnetic) are reliably identified, and the operation of the medical imaging device 2 is, for example, interrupted.

The bit error rate may be measured by the bit error registration unit 8 via, for example, an error counter being increased by a fixed increment in the case of an error and being reduced by 1 in the error-free case. If this counter exceeds an adjustable threshold, a warning may be output, or the operation of the device may be interrupted.

Advantages offered by one or more of the present embodiments are the ability to draw conclusions about elevated interference fields present in the surroundings of the device on the basis of evaluating the bit error rate and minimize the risk of a malfunction of the medical imaging device on the basis of the interference fields by way of the subsequent methods.

Although the invention is illustrated and described more closely by the exemplary embodiments, the invention is not restricted by the disclosed examples. Other variants may be derived herefrom by a person skilled in the art without departing from the scope of protection of the invention. For example, the invention may also be used in non-medical applications.

The elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A medical imaging device comprising:
a contactless data transmission apparatus configured to register acting interference fields, the contactless data transmission apparatus comprising:
a bit error registration unit configured to:
establish a bit error rate of the contactless data transmission apparatus caused by the acting interference fields; and
output a warning signal when a predeterminable threshold of the bit error rate is exceeded.

2. The medical imaging device of claim 1, further comprising an indication unit configured to represent the warning signal optically, acoustically, or optically and acoustically.

3. The medical imaging device of claim 1, further comprising a switching-off unit configured to interrupt operation of the medical imaging device when the warning signal occurs.

4. The medical imaging device of claim 1, wherein the contactless data transmission device comprises a stationary unit and a rotating unit, between which data is transmitted contactlessly.

5. The medical imaging device of claim 1, wherein the medical imaging device is a computed tomography scanner or a magnetic resonance imaging scanner.

6. The medical imaging device of claim 2, further comprising a switching-off unit configured to interrupt operation of the medical imaging device when the warning signal occurs.

7. The medical imaging device of claim 2, wherein the contactless data transmission device comprises a stationary unit and a rotating unit, between which data is transmitted contactlessly.

8. The medical imaging device of claim 3, wherein the contactless data transmission device comprises a stationary unit and a rotating unit, between which data is transmitted contactlessly.

9. The medical imaging device of claim 2, wherein the medical imaging device is a computed tomography scanner or a magnetic resonance imaging scanner.

10. The medical imaging device of claim 3, wherein the medical imaging device is a computed tomography scanner or a magnetic resonance imaging scanner.

11. The medical imaging device of claim 4, wherein the medical imaging device is a computed tomography scanner or a magnetic resonance imaging scanner.

12. A method for operating a medical imaging device comprising a contactless data transmission, the method comprising:
registering bit errors in the contactless data transmission caused by acting interference fields;
establishing a bit error rate; and
emitting a warning signal when a predeterminable threshold of the bit error rate is exceeded.

13. The method of claim 12, further comprising optically, acoustically, or optically and acoustically indicating the warning signal.

14. The method of claim 12, further comprising interrupting the operation of the medical imaging device when the warning signal occurs.

15. The method of claim 13, further comprising interrupting the operation of the medical imaging device when the warning signal occurs.

16. The method of claim 12, wherein the data transmission is performed between units that are moved relative to one another.

17. The method of claim 13, wherein the data transmission is performed between units that are moved relative to one another.

18. The method of claim 14, wherein the data transmission is performed between units that are moved relative to one another.
